(19) United States
(12) Patent Application Publication
(10) Pub. No.: US 2002/0042423 A1
(43) Pub. Date: Apr. 11, 2002

(54) METHODS FOR IDENTIFYING COMPOUNDS THAT BIND TO HLA MOLECULES AND USE OF SUCH COMPOUNDS AS HLA-AGONISTS OR ANTAGONISTS

(76) Inventors: JOHN R. RICHERT, CHEVY CHASE, MD (US); MING LIU, FALLS CHURCH, VA (US); XIONG-WU WU, FALLS CHURCH, VA (US); SHAOMENG WANG, MCLEAN, VA (US); NIKLAS KOHLER, BRAUNSCHWEIG (DE); DAXU YIN, BALTIMORE, MD (US)

Correspondence Address: ROBIN L. TESKIN, SHAW PITTMAN, 2300 N STREET, N.W., WASHINGTON, DC, 20037-1128, US

(21) Appl. No.: 09/301,339

(22) Filed: Apr. 29, 1999

Related U.S. Application Data
(60) Provisional application No. 60/083,426, filed on Apr. 29, 1998.

(51) Int. Cl. A61K 31/505; A61K 31/47; A61K 31/13; A61K 31/16

(52) U.S. Cl. 514/259; 514/260; 514/307; 514/579

(57) ABSTRACT

A novel method for identifying compounds which bind HLA molecules and which can be used as HLA agonists or antagonists is provided. These compounds are useful especially in the treatment of autoimmune diseases, transplantation, graft-vs-host disease, and more particularly multiple sclerosis.

### BACKGROUND OF THE INVENTION

1. Field of the Invention

The present invention relates to a novel method of identifying compounds suitable for prevention or treatment of diseases where HLA-restrictive antigen-specific immune responses play a significant role. Examples thereof include autoimmune diseases, graft versus host disease, transplant rejection, and in particular multiple sclerosis. The present invention also relates to specific compounds identified by this novel method which inhibit in particular the interaction of myelin basic protein (MBP) to HLA molecules.

2. Background of the Invention

A portion of the immune response in mammals is dependent upon the body's ability to recognize and respond to protein antigens. These protein antigens are known to bind to the Major Histocompatability Complex (MHC) molecules expressed on the surface of certain cells. T-cells, in turn, are presented processed protein antigen by the MHC complex and an immune response is created. The MHC molecules are classified as either Class 1, which are involved in the creation of a T killer cell response, and Class II, which present antigen to T helper cells, thereby participating in the production of antigen specific antibodies. The Class II MHC molecules have been further identified in humans as being HLA-DP, -DQ or -DR.

Of particular interest in multiple sclerosis is the HLA-DR locus. In multiple sclerosis (MS) CD4+ T-cells are thought to play a pivotal pathological role in mediating an autoimmune attack against myelin components. Myelin Basic Protein (MBP) is one of several potential autoantigens in the disease. It is believed that MS-associated HLA molecules bind certain immunodominant peptides and these complexes in turn trigger (auto)reactive T-cell receptors (TCRs).

Two immunodominant MBP T-cell epitopes have been identified; MBP 152-165, which is sometimes restricted by DR13 (DRα,β*1301), and MBP 83-97, which is often restricted by DR2 (DRα,β*1501). The amino acid residues that anchor these two MBP peptides to HLA-DR, and which likely interact with binding pockets in the floor of the DR antigen binding groove, have also been defined. Thus, the components of the resultant trimolecular complex, i.e., the above-identified disease-associated HLA molecule, the immunodominant MBP peptide, and the TCR are anticipated targets for new, more specific therapies.

In the past, scientists have attempted to interfere with MBP recognition by autoreactive T-cells by two different approaches. The first is through the use of MHC-specific monoclonal antibodies (Abs). The second approach is through the use of peptides with high binding affinity to the MHC, which compete for specific MHC binding sites, thereby blocking activation. Both approaches are less than optimal. A significant problem, however, is instability attributable to proteinase degradation upon in vivo administration of both Abs and peptides. Also, because these moieties are themselves proteinaceous, there exists as the possibility that they will themselves elicit an anti-idiotypic or anti-peptide immune response. In addition, altered peptide ligands have the tendency to alter T-cell function and are more peptide-specific than HLA-specific.

For example, Mokhterian, Foroozan, Clin. Imunol. Immunopathol., 44:308-317 (1988), reported that the addition of specific anti Ia antibody blocked the antigen-specific proliferation of T-cells and inhibited the transfer of both acute and relapsing experimental allergic encephalomyelitis (EAE) in SJL/J mice, an accepted animal model for study of multiple sclerosis. Further, administration of specific anti-I-A antibody to mice actively immunized with a mouse spinal cord homogenate has been reported to block the induction of EAE. (Steinman et al, Proc. Natl. Acad. Sci. USA, 78:7111 (1981)). Therefore, it would be useful if improved compounds could be developed useful for modulating specific HLA-antigen interactions. In particular, it would be helpful if compounds having improved selectivity, solubility and stability could be obtained.

### Definitions

The following abbreviations have the following definitions: (Abs) Antibodies; (HLA) Histocompatability Lymphocyte-A System; (MBP) Myelin Basic Protein; (MHC) Major Histocompatability Complex; (MS) Multiple Sclerosis; (TCRs) T-cell Receptors; (%) percentage; (α) alpha; (β) beta, (CD4+) T-cell marker specific to helper T-cells, receptor; (mM) millimolar; (DMEM) Dulbecco's Modified Eagle Media; (U/ml) units per milliliter; and (ELISA) Enzyme Linked Immunosorbent Assay.

### SUMMARY AND OBJECTS OF THE INVENTION

It is an object of the invention to provide a method for identifying compounds that selectively affect (agonize or antagonize) the interaction of HLA molecules and antigen.

It is a more specific object of the invention to provide a method for identifying compounds that selectively affect (agonize or antagonize) interaction of HLA molecules and antigen that combines computer modeling methods and in vitro binding assays.

It is another specific object of the invention to provide novel therapeutic methods that provide for modulation of specific HLA-antigen interactions comprising that administration of a compound identified by the novel methods provided herein.

It is a more specific object of the invention to provide novel methods for treating disorders wherein inhibition of HLA-antigen interactions is therapeutically desirable, in particular transplantation, graft-vs-host disease and autoimmune disorders involving the administration of at least one compound identified according to the novel methods provided herein.

It is an even more specific object of the invention to modulate HLA-DR1301-antigen interactions for treatment or prevention of diseases associated therewith, in particular multiple sclerosis, by the administration of at least one compound identified by the novel methods reported herein.

### BRIEF DESCRIPTION OF THE DRAWINGS

FIG. 1 displays a 3D model of the HLA-DR1301 molecule produced by homology modeling;

FIG. 2 displays a 3D model showing the binding pockets of DR1301 occupied by residue 162 and residue 154 of MBP;

FIG. 3 illustrates the binding of HLA-DR1301 and HLA-DR1501 by the initial lead compound #105;

FIG. 4 displays the binding of compound #105 to HLA-DR1301;

FIG. 5 displays a computer generated 3D visualization of compound #105 and analogs #6 and #14, #6 showing a greater specificity for DR1301 than #105, while #14 is inactive;

FIG. 6 compares the ability of several compounds identified by use of MCDOCK to bind to and block HLA-DR1301 and DR1501;

FIGS. 7A-7F contain structures for analogs of the initial lead compound identified according to the inventive methods;

FIG. 8 contains structures of the lead compound #105 and preferred analogs;

FIG. 9 contains the results of a functional assay which evaluates the effect of the lead compound #105 on IL-2 production by TCR transfectants stimulated by MBP peptide/HLA-DR1301 or MBP peptide/HLA-DR1501; and

FIG. 10 contains the results of a competition binding assay using biotinylated MBP peptide and different concentrations of an analog of the initial lead compound #105 (analog 6). The results contained in the Figure indicate that this analog (analog 6 of compound #105) specifically competes with MBP peptide for binding to purified DR1301 molecules.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Through the use of computer simulation, the three dimensional (3D) surface structure of an HLA molecule can be used as a target for predicting drug design In this way blocking compounds can be found through computer assisted searches of databases. Compounds which contain the best predicted fit can then be visually inspected and tested under in vitro and in vivo conditions. This method also allows for the tinkering of the compound's structure to allow for optimal binding capacity, i.e., by testing the activity of analogs of the identified compounds in in vitro assays.

In its broadest embodiment, the present invention provides a method for computational processing of a database containing three-dimensional structures of a large number of chemical compounds to identify compounds having high predicted binding affinity to a host molecule. The predicted binding affinity is validated through in vitro testing. One or more of the compounds having a binding affinity validated in vitro are further tested in vivo to provide a group of pharmacophores capable of having therapeutic activity involving the host molecule.

Computationally predicting a compound's binding affinity to a host molecule involves utilizing the three dimensional (3-D) structures of the host and the compound. As indicated above, the 3-D structure of the compound is obtained from a database of chemical compounds. The 3-D structure of the host protein can also be obtained from a protein database. However, in spite of important increases in the number of proteins having available 3-D structures, that number only covers a very small fraction of proteins having known biological function. Therefore, the invention includes a method for modeling the 3-D structure of the host protein, when such structure is not available.) Modeling the 3-D structure of the host protein includes obtaining the primary and secondary structures of the protein. Screening a database containing proteins having known 3-D structures, and retrieving from the database the structure of a protein having primary and/or secondary structures having a high degree of homology with the primary and/or secondary structures of the host molecule. The screening and selection methods are performed using one of the available homology screening computer programs. One example of a computer program capable of identifying a homologous protein of known 3-D structure is provided in the software package BLAST. BLAST can be accessed at http://www.ncbi.nlm.nih.gov/BLAST/. The methodology utilized in BLAST is described in “Protein sequence similarity searches using patterns as seeds”, by Zheng Zhang, Alejandro A. Schffer, Webb Miller, Thomas L. Madden, David J. Lipman, Eugene V. Koonin, and Stephen F. Altchsul (1998), Nucleic Acids Res. 26:3986-3990, the contents of which are incorporated herein by reference in their entirety.

A template 3-D structure of the host protein is obtained through the program MODELLER. MODELLER can be obtained from Professor Andrej Sali, the Rockefeller University, 1230 York Avenue, New York, N.Y. 10021-6399. The methodology utilized in MODELLER is described in “Evaluation of comparative protein modeling by MODELLER” by Sali, A., Potterton, L., Yuan, F., van Vlijmen, H., & Karplus, M. (1995). Proteins, 23, 318-326, the contents of which are incorporated herein by reference in their entirety.

In forming a template 3-D structure of the host protein, each atom in of the backbone of the protein is assigned a position corresponding to the equivalent backbone atom of the homologous protein. Similarily, each atom of a side chain of the host protein having an equivalent side chain in the homologous protein is assigned the position corresponding to the position of the atom in the equivalent side chain of the homologous protein. The atom positions for the side chains not having an equivalent in the homologous protein are determined by constructing the side chain according to preferred internal coordinates and attaching the side chain to the backbone of the host protein. The template structure thus obtained is refined by minimizing the internal energy of the template protein. During the refinement, the positions of the atoms of the side chains having no equivalents in the homologous protein are adjusted while keeping the rest of the atoms of the template protein in a fixed position. This allows the atoms of the constructed side chains to adapt their positions to the part of the template structure determined by homology. The full template structure is then minimized (relaxed) by allowing all the atoms to move. Relaxing the template 3-D structure of the protein eliminates unfavorable contacts between the atoms of the protein and reduces the strain in the template 3-D structure.

The minimization of the energy function associated with the template structure can be performed by any minimization technique. A preferred minimization technique involves simulated annealing. This technique is incorporated in numerous commercial and non-commercial computer programs. One such computer program is included in the software package CHARMM. CHARMM can be obtained either from Dr. Martin Karplus at the Harvard University for academic users or from the Molecular Simulation Inc., San Diego, Calif. The simulated annealing methodology incorporated in CHARMM is described in “A program for macromolecular energy minimization, and dynamics calculations” by Brooks, B. R., Bruccoleri, R. E., Olafson, B. D., States, D. J., Swaminathan, S., and Karplus, M., J. Comp. Chem. 1 (1983) 187, the contents of which are incorporated herein by reference in their entirety.

Based on the refined structure of the host protein, a host-guest complex is formed by disposing a compound from the database in a receptor site of the protein.

The structure of the host-guest complex is defined by the position occupied by each atom in the complex in a three dimensional referential. The position of each atom is defined by a set of three coordinates in the referential. The structure of the host-guest complex is incorporated in a computer program capable of determining the degree of geometrical fit between the guest and the host in the complex. Programs based on shape complementarity can effectively rank guest-host complexes based on the geometrical fit between the host and the guest. A preferred program for ranking guest-host complexes based on the geometrical fit is provided in the software package DOCK. DOCK can be obtained from Dr. Irwin Kuntz at the Department of Pharmaceutical Chemistry, University of California at San Francisco, USA. The shape complementarity methodology of DOCK is described in “Critical evaluation of search algorithms used in automated molecular docking” by Ewing, T. J. A., and Kuntz, I. D. J. Comput. Chem. 18(9): 1175-1189, 1997, the contents of which are incorporated herein by reference in their entirety.

A group of compounds is extracted from the compound database for further processing based on their geometry fit rank. The compounds in the group have a guest-host complex geometrical fit of a predetermined rank or higher. The number of compounds in the geometry fit group is generally a small fraction of the total number of compounds in the database.

For each compound in the geometry fit group, a predicted binding affinity to the receptor site of the host protein is determined by minimizing an energy function describing the interactions between the atoms of the compound and those of the protein. The minimization of the energy function is conducted by changing the position of the compound such that a guest-host complex structure corresponding to a minimum of the energy function is obtained.

The energy function includes energy terms describing non-bonded interactions between the atoms of the compound and those of the protein. The non-bonded energy terms include a term for atom-atom Van der Waals interactions and a term for charge-charge electrostatic interactions. The energy function does not include constraints on torsional degrees of freedom of the compound which provides greater flexibility in changing the position and conformation of the compound in the receptor site of the protein. A minimum energy value is obtained for each compound-protein complex.

Allowing for torsional flexibility in refining the structure of the complex greatly enhances the accuracy of the predicted binding energy of the complex. In this regard, a flexible compound can adopt a larger number of conformations inside the receptor site, thus allowing for probing a larger number of complex structures. Increasing the number of probable complex structures increases the probability of identifying a global minimum of the energy function. That is, a minimum having an energy lower than the energy associated with one or more other identified minima of the energy function (local minima). Identifying a global minimum for a given complex is greatly advantageous in that a more accurate predicted binding affinity is obtained for the complex. Increasing the accuracy of the predicted binding affinity increases the accuracy in energy based discrimination between the compounds of the geometry fit group, thus providing the best candidates for in vitro testing.

Several computational techniques have been previously used in adjusting the position of a guest in relation to a host. However, conventional programs based on those techniques do not provide satisfactory torsional flexibility in moving the guest within the receptor site of the host. Therefore, a new approach is provided for effectively including torsional energy in refining the position of the compound in the complex. The new approach is implemented in the computer program MCDOCK, a copy of which can be obtained from Dr. Shaomeng Wang at the Georgetown University Medical Center.

The complex energy minimization employs a non-conventional Monte Carlo simulation technique. The methodology incorporated in MCDOCK is described in “MCDOCK: A Monte Carlo simulation approach to the molecular docking problem” by Ming Liu and Shaomeng Wang, to be published in Journal of Computer-Aided Molecular Design, the contents of which are incorporated herein by reference in their entirety.

MCDOCK provides a minimization method based on a non-conventional Monte Carlo simulation technique which allows greater probability to reach a global energy minimum. In particular, the program only constrains the bonds and bond angles describing the structure of the guest host complex. Otherwise, the atoms are allowed to move freely in a force field determined by an energy function formed by Van der Waals and electrostatic terms only. This flexibility allows the guest to adopt various conformations within the receptor site of the host and thus explore a larger portion of the receptor site. This in turn allows the exploration of global minima, which improves the equality of the energy based binding affinity prediction.

The compounds in the geometry fit group are processed through MCDOCK such that for each compound, a compound-protein complex of minimum “MCDOCK” energy is determined. The compounds are then ranked according to the minimum energy obtained. A subgroup of compounds associated with complexes having a minimum energy lower than a predetermined energy value is formed. The number of compounds in the subgroup is also a small fraction of the total number of compounds in the geometry fit group.

The binding information associated with each compound in the subgroup is further refined by displaying on a computer screen an image of the complex structure of minimum energy. Displaying the compound-protein complex is conducted through one of the conventional chemical structure graphic visualization tools. A preferred graphic visualization tool is provided in the software package QUANTA (MOLECULAR SIMULATIONS, San Diego, Calif.).

The displayed complexes are visually examined to form a group of candidate compounds for in vitro testing. For example, the complexes are inspected for visual determination of the quality of docking of the compound into the receptor site of the protein. Visual inspection provides an effective basis for identifying compounds for in vitro testing. It should be noted that such visual inspection is impractical without the effective pruning of the compounds of the initial database provided by the pruning based on the combination of the geometry fit and complex energy minimization. Therefore, the number of compounds in the group discarded in the visual pruning step is much smaller than the number of compounds discarded in the geometry fit and energy based pruning.

After putative binding compounds have been identified, the ability of such compounds to specifically bind to a particular receptor moiety, e.g., a specific HLA molecule, will be confirmed in vitro.

Methods for determining whether a compound binds to a particular receptor, i.e., receptor binding assays are well known in the art. In particular, this can be effected by use of competition assays. In general, this will involve providing a source of the particular receptor, e.g., HLA molecule, a moiety known to interact with such receptor, e.g., peptide, and a compound, the receptor binding of which is to be tested. Compounds which bind the receptor will inhibit the binding of the other moiety, e.g., peptide, that is known to specifically bind said receptor.

Also, in the case of putative HLA-binding compounds, these compounds can be tested in functional assays which test the ability of these compounds to affect (block) antigen presentation by HLA-transfected antigen-presenting cells to T-cell receptor (TCR)-transfected T cells.

As discussed infra, in the examples, a number of compounds were identified that putatively bound HLA-DR1301, at a site at which this molecule interacts with MBP. These putative HLA binding compounds identified by computational methods were then evaluated in biological assays that tested toxicity to specific cell lines. This was effected because of the desired in vivo application of such compounds. The compounds which were found to be non-toxic were then tested to evaluate whether they specifically bound to HLA-DR1301, and in functional assays that assess whether the compound inhibits IL-2 production by T cells in the presence of MBP. Functional assays for identifying the effects of compounds on HLA-antigen binding and T cells are well known in the art. The present inventors in particular utilized a biological assay which measures the effect on antigen presentation by HLA-transfected antigen-presenting cells to T cell receptor (TCR)-transfected cells, in the presence of the putative HLA binding compound and the antigen that normally binds said HLA molecule (e.g., MBP). If antigen presentation is inhibited, IL-2 secretion is reduced (inhibited). Therefore, the ability of a putative compound to agonize or antagonize HLA-antigen interactions can be assessed based on its effect on IL-2 secretion.

Those compounds that exhibit activity in the functional assay will be tested in a receptor binding assay that determines the selectivity and affinity of the binding of the compound to a particular HLA molecule, e.g., HLA-DR1301. For example, this can be determined by use of competitive binding assays which measure binding of the compound to an HLA molecule in the presence of a labeled compound that normally binds the receptor, e.g., biotinylated MBP.

As discussed above, the present invention preferably will identify non-proteinaceous, organic small molecules that specifically bind to specific sites on MHC (HLA) molecules that interact with antigens, e.g., autoantigens, or themselves serve as transplantation antigens. This is significant as HLA molecules themselves serve as triggers of transplant rejection reactions.

It is well known that an important portion of the immune response of mammals including humans, involves the interaction of antigens with Major Histocompatability Complex (MHC) molecules which are expressed by specific immune cells. These immune responses can be classified into two groups, Class I responses, which involve the interaction of MHC/antigen complexes with killer T-cells, and Class II responses, which involve the interaction of MHC/antigen complexes with helper T-cells, which in turn are involved in the production of antigen-specific antibodies.

In humans, MHC molecules are referred to as HLA molecules. Moreover, Class II MHC molecules in humans are further classified into various sub-groups, i.e., HLA-DP, HLA-DQ, and HLA-DR.

Most MHC (HLA)-antigen interactions are beneficial to the well being of a mammal (human) as they are involved in protecting a subject from infectious agents such as viruses, bacteria, or other pathogens. However, in some instances, these interactions can be deleterious to a subject. A particular situation wherein HLA-antigen mediated immune responses can be highly adverse to a subject's well being is in autoimmune diseases. Essentially, in such diseases, a subject reacts to specific autoantigens as if they were foreign or heterologous to the subject, and elicits autoantigen mediated immune reactions characterized by the production of autoantibodies, i.e., antibodies specific to “self”, and T-cell responses which may result in pathological effects.

Autoimmune diseases can be classified into two main groups, antibody-mediated autoimmune diseases, and T-cell mediated autoimmune diseases.

Antibody mediated autoimmune diseases are autoimmune diseases wherein autoantibodies are significantly involved in pathology. Examples thereof include systemic lupus erythematosus, glomerulonephritis (Goodpasture's syndrome), autoimmune hemolytic anemia, autoimmune thrombocytopenic purpura (ITP), pemphigus vulgaris, bullous pemphigoid, myasthenia gravis, Graves' disease, insulin-resistant diabetes mellitus, and pernicious anemia, among others. In such diseases, autoantibodies may elicit clinical symptoms including nephritis, proteinuria, hemolysis, platelet deficiency, muscle weakness, arthritis, inflammatory responses, among others.

By contrast, T-cell mediated autoimmune diseases are autoimmune diseases wherein antigen-specific T-cells, or other non-antibody producing cells, are involved in pathology. Examples thereof include insulin-dependent (type I) diabetes mellitus, experimental allergic encephalomyelitis, multiple sclerosis, experimental allergic neuritis, experimental autoimmune myocarditis, some forms of Graves' disease, and others.

In these diseases, T-cells of the CD4⁺ or CD8⁺ subset secrete cytokines that give rise to DTH reactions that may result in tissue injury elicited by activated macrophages and cytotoxic T-lymphocytes.

Also, some autoimmune diseases can be considered “mixed” in that autoantigens elicit both antibodies which are involved in pathology and T-cell mediated pathological responses. An example thereof is rheumatoid arthritis which is characterized by large quantities of circulating autoantibodies (IgM specific for Fc portion of IgGs) which are associated with T-cell mediated tissue destruction, particularly of the joints.

Another situation wherein HLA-antigen interactions may be undesirable is in transplantation. Typically, subjects who received transplanted cells, tissues or organs receive immunosuppressants, in particular cyclosporin, the purpose of which is to suppress the host's reaction to foreign antigens expressed by the transplanted cells, tissue or organ. However, such immunosuppressants are non-selective, that is they suppress immune reactions to different antigens, including antigens wherein immune responses are desirable, e.g., pathogens.

Thus, the present invention is advantageous in that it provides small molecules which selectively interact with particular HLA molecules, and more specifically that interact with specific binding sites on particular HLA molecules. Thereby, these small molecules should inhibit or actively intervene in antigen reactions which involve these specific HLA molecules, and more specifically antigen reactions which involve these particular HLA binding sites.

The small molecules identified by the subject screening method can be used to modulate (inhibit or enhance) immune reactions that are elicited by the interaction of HLA molecules with antigen. As noted, HLA-antigen interactions are involved in many diseases. In particular, the subject small molecules may be used in the treatment or prevention of autoimmune diseases, both T and B-cell mediated autoimmune diseases, transplantation, and graft-versus-host disease.

Specifically, the present inventors have identified non-proteinaceous small molecule compounds that bind to HLA-DR13 (DRα,β*1301) and HLA-DR2 (DRα,β*1501), at sites which respectively interact with immunodominant myelin basic protein (MBP) epitopes, i.e., MBP 152-165 and MBP 83-97. It has been reported that these immunodominant peptides play a pivotal role in the elicitation of pathological CD4⁺ T-cell responses in multiple sclerosis, specifically in mediating an autoimmune attack against myelin compounds. Thus, these small molecules should be useful in treating or preventing multiple sclerosis or other immune reactions and diseases wherein these or homologous HLA binding sites play a role in antigen interactions that are involved in disease pathology. In this regard, it is noted that HLA-DR1301, which binds to the identified small molecules has high sequence homology (sequence identity) with HLA-DR1, i.e., 100% homology in the α-chain and 80% homology in the β chain. Based on this homology, it is reasonable to expect that the subject molecules will potentially interact with other HLA molecules, and thereby modulate other HLA-antigen interactions. The selectivity of the small molecules can be tested by determining whether the subject small molecules bind to other HLA molecules, e.g., expressed on the surface of other HLA transfectants.

In particular, the present inventors have discovered that compounds having the generic formula set forth below specifically interact with HLA-DR1301 and HLA-DR1501 molecules:

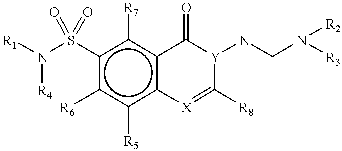

wherein R₁ and R₂ are selected from phenyl, substituted-phenyl, benzyl, substituted-benzyl (e.g., substituted with one or more halogens, hydroxyl, metals, nitro, SO₂, etc.), or any 5- or 6-membered aromatic ring system which may contain one or more heteroatoms, e.g., oxygen, sulfur, nitrogen, R₃ and R₄ are selected from the group consisting of H, phenyl, substituted-phenyl, benzyl, substituted-benzyl (e.g., substituted with one or more halogens, hydroxyl, metals, nitro, SO₂, etc.), and other aromatic ring systems, preferably 5- to 7-membered ring systems, alkyl (preferably C₁ to C₁₀), alkoxy (preferably C₁-C₁₀) halogen, SO₃M (where M is H or alkyl) (preferably C₁-C₁₀), amide, or COOR, where R₁ is H or alkyl (preferably C₁-C₁₀);

R₅, R₆, R₇ and R₈ are the same or different and are selected from H, halogen (F, Cl, Br, I), alkyl (preferably C₁-C₁₀), alkoxy (preferably C₁-C₁₀), amide, nitro, amine, cycloalkyl (preferably C₁-C₁₀), nitroso, hydroxyl, ether, ester, sulfonic acid, alkenyl (preferably C₁-C₁₀), allyl (preferably C₁-C₁₀), and X and Y are selected from nitrogen and carbon and may be the same or different.

A listing of preferred compounds identified according to the invention is reproduced below:

Lead Compound #105

2-{[4-(acetylamino)phenyl]amino}-N-[6-({[4-(acetylamino)phenyl]amino}sulfonyl)-4-oxo(3-hydroquinazolin-3-yl)]acetamide;

Analog 1

N-{2-methyl-4-oxo-6-[(phenylamino)sulfonyl](3-hydroquinazolin-3-yl)}-2-(phenylamino)acetamide;

Analog 2

2-[(2-methoxyphenyl)amino]-N-(6-{[(2-methoxyphenyl)amino]sulfonyl}-2-methyl-4-oxo(3-hydroquinazolin-3-yl))acetamide;

Analog 3

2-[(4-methoxyphenyl)amino]-N-(6-{[(4-methoxyphenyl)amino]sulfonyl}-2-methyl-4-oxo(3-hydroquinazolin-3-yl))acetamide;

Analog 4

2-[(2-chlorophenyl)amino]-N-(6-{[(2-chlorophenyl)amino]sulfonyl}-2-methyl-4-oxo(3-hydroquinazolin-3-yl))acetamide;

Analog 5

2-[(4-chlorophenyl)amino]-N-(6-{[(4-chlorophenyl)amino]sulfonyl}-2-methyl-4-oxo(3-hydroquinazolin-3-yl)acetamide;

Analog 6

2-[(2,4-dichlorophenyl)amino]-N-(7-{[(2,4-dichlorophenyl)amino]sulfonyl}-1-oxo(2-2-hydronaphthyl))acetamide;

Analog 7

2-[(2,6-dichlorophenyl)amino]-N-(7-{[(2,6-dichlorophenyl)amino}sulfonyl}-1-oxo(2-2-hydronaphthyl))acetamide;

Analog 8

2-({[N-(6-{[(2- carboxyphenyl)amino]sulfonyl}-4-oxo-3-hydroquinazolin-3-yl)carbamoyl]methyl}amino)benzoic acid;

Analog 9

2-[(2-nitrophenyl)amino]-N-(6-{[(2-nitrophenyl)amino]sulfonyl}-4-oxo(3-hydroquinazolin-3-yl)acetamide;

Analog 10

2-[(2-acetylphenyl)amino]-N-(6-{[(2-acetylphenyl)amino]sulfonyl}-4-oxo(3-hydroquinazolin-3-yl))acetamide;

Analog 11

N-{4-oxo-6-[(1,3-thiazol-2-ylamino)sulfonyl](3-hydroquinazolin-3-yl)}-2-(1,3-thiazol-2-ylamino)acetamide;

Analog 12

4-({[N-(6-{[bis(4-sulfophenyl)amino]sulfonyl}-4-oxo(3-hydroquinazolin-3-yl))carbamoyl]methyl}(4-sulfophenyl)amino)benzenesulfonic acid;

Analog 13

3-{[(4-chlorophenyl)sulfonyl]amino}-6-{[(2-methoxyphenyl)amino]sulfonyl}-3-hydroquinazolin-4-one;

Analog 14

3-{[(4-iodophenyl)sulfonyl]amino}-6-{[(4-methoxyphenyl)amino]sulfonyl}-3-hydroquinazolin-4-one;

Analog 15

N-{4-oxo-6-[(2-pyridylamino)sulfonyl](3-hydroquinazolin-3-yl)}-2-(2-pyridylamino)acetamide.

The most preferred compound, i.e., lead compound, identified by the invention is analog #6, 2-[(2,4-dichlorophenyl)amino]-N-(7-{[(2,4-dichloro-phenyl)amino]sulfonyl}-1-oxo(2-2-hydronaphthyl))acetamide. Moreover, the invention further embraces the use of isomers and pharmaceutically acceptable salts of the subject compounds and their derivatives.

The subject compounds can be used to treat any condition wherein modulation of HLA-antigen interactions is therapeutically beneficial. The invention embraces the use of compounds which function as HLA agonists or antagonists. Typically, the compound will be used to antagonize, i.e., inhibit, HLA-antigen interactions, which are involved in pathological responses, e.g., B and T-cell mediated autoimmune diseases selected from the group consisting of psoriasis; dermatitis; systemic scleroderma and sclerosis; responses associated with inflammatory bowel disease; Crohn's disease, ulcerative colitis; respiratory distress syndrome; adult respiratory distress syndrome (ARDS); dermatitis; meningitis; encephalitis; uveitis; colitis; glomerulonephritis; allergic conditions; eczema; asthma; conditions involving infiltration of T-cells and chronic inflammatory responses; atherosclerosis; leukocyte adhesion deficiency; rheumatoid arthritis; systemic lupus erythematosus (SLE); diabetes mellitus; multiple sclerosis; Reynaud's syndrome; autoimmune thyroiditis; allergic encephalomyelitis; Sjorgen's syndrome; juvenile onset diabetes; immune responses associated with acute and delayed hypersensitivity mediated by cytokines and T-lymphocytes; tuberculosis; sarcoidosis; polymyositis; granulomatosis; vasculitis; pernicious anemia (Addison's diseases); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; idiopathis thrombocytopenic purpura (ITP) and autoimmune thrombocytopenia.

In the preferred embodiment, the treated autoimmune disease will comprise multiple sclerosis.

Also, compounds identified according to the invention can be utilized in other conditions wherein immunosuppression is desirable, e.g., in transplant recipients and to prevent or alleviate graft-vs-host disease. For example, an inventive compound may be administered to recipients of cells, allogeneic or xenogeneic tissues or organs such as the heart, lung, liver, pancreatic islets, kidney, neural cells, bone marrow, spleen, bone, skin, stomach, intestine, et seq.

Moreover, compounds according to the invention may be utilized during gene or cell therapy, in order to suppress HLA-antigen mediated immune reactions against the vector or cell used for gene or cell therapy. This is significant as a prevalent problem that precludes effective gene and cell therapy is the often short in vivo half life of such cells and vectors because of host immune responses.

Also, the subject compounds may be administered during other therapies that involve the administration of potentially antigenic therapeutics, e.g., growth factors, hormones, antibodies, toxins, etc. Thereby, the efficacy of such therapeutics can be prolonged.

Further, the subject compounds may be administered in conjunction with other immune modulators and suppressants, e.g., cytokines, anti-cytokine antibodies, anti-cytokine receptor antibodies, e.g., anti-TNF, anti-TNF receptor, cyclosporin, CD40-ligand agonists and antagonists, CD40 and soluble forms thereof, methotrexate, etc.

Also, the subject compounds can be used as HLA agonists and enhance immune reactions elicited by HLA-antigen interactions. In particular, HLA agonists may be useful in the treatment of cancer, parasitic diseases, viral diseases, or other disorders wherein the host immune responses may be suppressed. For example, HLA agonists may be identified which enhance HLA-antigen interactions that are involved in anti-tumor or anti-viral T cell mediated immune responses.

The subject compounds can be administered by any pharmaceutically acceptable means, e.g., orally, parenterally, subcutaneously, intrapulmonarily, intranasally, rectally, topically, et seq. Parenteral methods include intramuscular, intravenous, intraarterial, intraperitoneal, and subcutaneous administration. Oral administration is typically preferred.

The subject compounds can be administered in a single or repeated dosages. Given the chronic nature of many autoimmune diseases and the desire to maintain transplants indefinitely, it is anticipated that chronic or prolonged administration will be preferred.

The subject compounds will be administered in a pharmaceutically acceptable formulation, which include by way of example tablets, liposomal formulations, injectable formulations, milks, creams, oil-in-water or water-in-oil emulsions, microcapsules, etc.

The effective dosage will vary typically from about 0.001 to 2000 mg/kg of body weight, more typically 0.01 to 200 mg/kg of body weight, and most typically will range from about 0.1 to 100 mg/kg of body weight.

Effective dosages will vary dependent upon factors including the particular compound, its HLA binding affinity and selectivity, the condition of the subject treated, mode of administration, and whether the compound is used alone or with other therapeutics.

The administered compound can be combined with known carriers and excipients used in drug formulations, e.g., buffers, such as phosphate, citrate and other organic acids, antioxidants, preservatives, diluents, tabletting materials, oils, polysaccharides, etc.

The compounds may be entrapped in microcapsules, in colloidal drug delivery systems such as liposomes, albumin microemulsions, nanoparticles, nanocapsules, or in macroemulsions. Suitable materials and methods for preparing pharmaceutical formulations are disclosed in Remington's Pharmaceutical Sciences, 16^{th} Edition, Osol, A. Ed. (1980).

Sustained release preparations may also be prepared. Suitable examples of sustained-release preparations include semipermeable matrices of solid hydrophobic polymers containing the subject HLA-agonist or antagonist compound, which matrices may be in the form of shaped articles, e.g., films or microcapsules. Examples of sustained-release matrices include polyesters, hydrogels, and various other polymers and co-polymers known in the pharmaceutical art.

The invention will now be described in more detail in the following Examples.

### EXAMPLE 1

### Computational Identification of Lead Compounds for Blocking the Receptor Site of HLA-DR1301

The 3-D structure of HLA-DR1301 is not available. Therefore, a search of the Protein Data Bank (PDB) is conducted through the program BLAST to identify a protein of known 3-D structure having a high degree of primary and secondary structure homology with HLA-DR1301. PDB can be accessed at http://www.rcsb.org/. DR1 is identified by BLAST as having a secondary structure homology to HLA-DR1301. DR1 and HLA-DR1301 have a 100% α-chain homology and a 80% β-chain homology.

A template structure of HLA-DR1301 is computationally modeled through the program MODELLER. Each atom in the backbone of HLA-DR1301 is assigned a position corresponding to the position of the equivalent atom in the 3-D structure of DR1. Similarly, each atom of a side chan of HLA-DR 1301 having an equivalent side chain in DR1 is assigned a position corresponding to the position of the atom in the equivalent side chain of DR1. The atoms of the side chains of HLA-DR 1301 not having equivalents in DR1 are determined by positioning the side chain according to its position in the amino acid sequence of HLA-DR 1301 and refining the template structure thus obtained. The refined template structure is then relaxed to reduce the strain which may be present in the refined template. The refining and relaxing of the HLA-DR 1301 model structure is conducted according to the procedures described above.

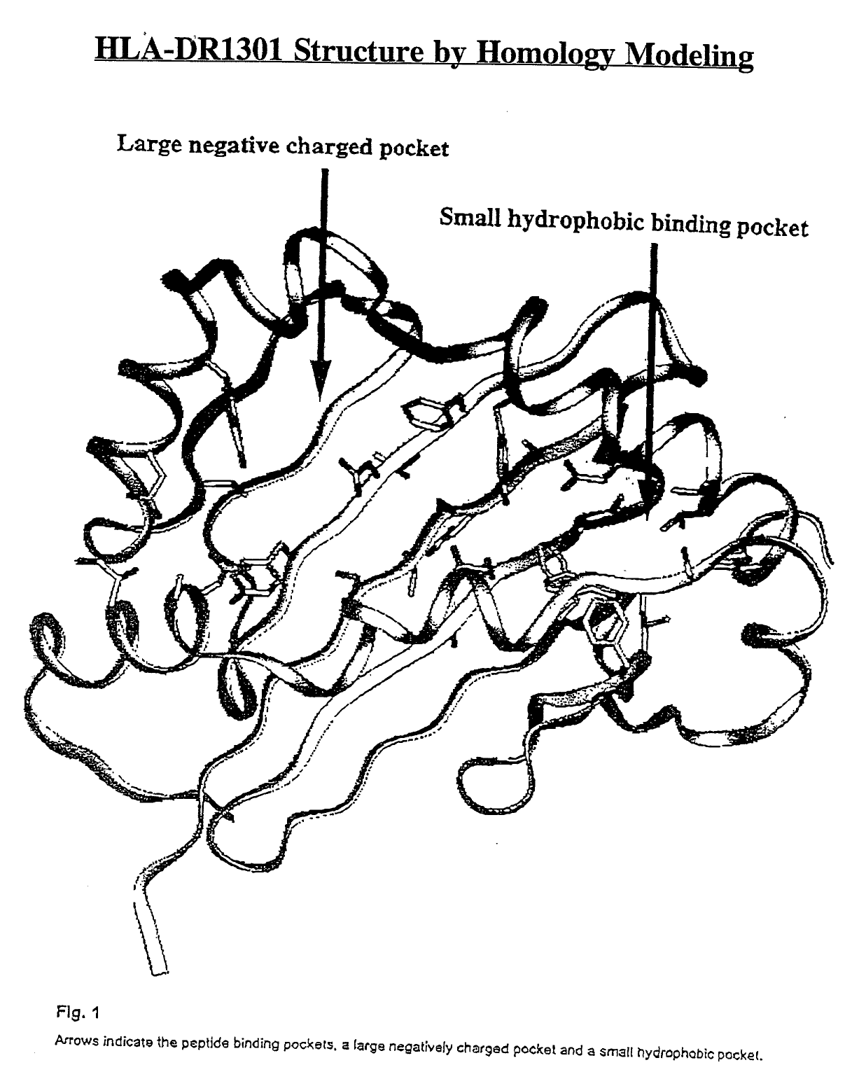

A 3-D model of the homology based HLA-DR1301 template is shown in FIG. 1. The figure shows a receptor site formed by two binding pockets. A large negatively charged pocket and a small hydrophobic pocket.

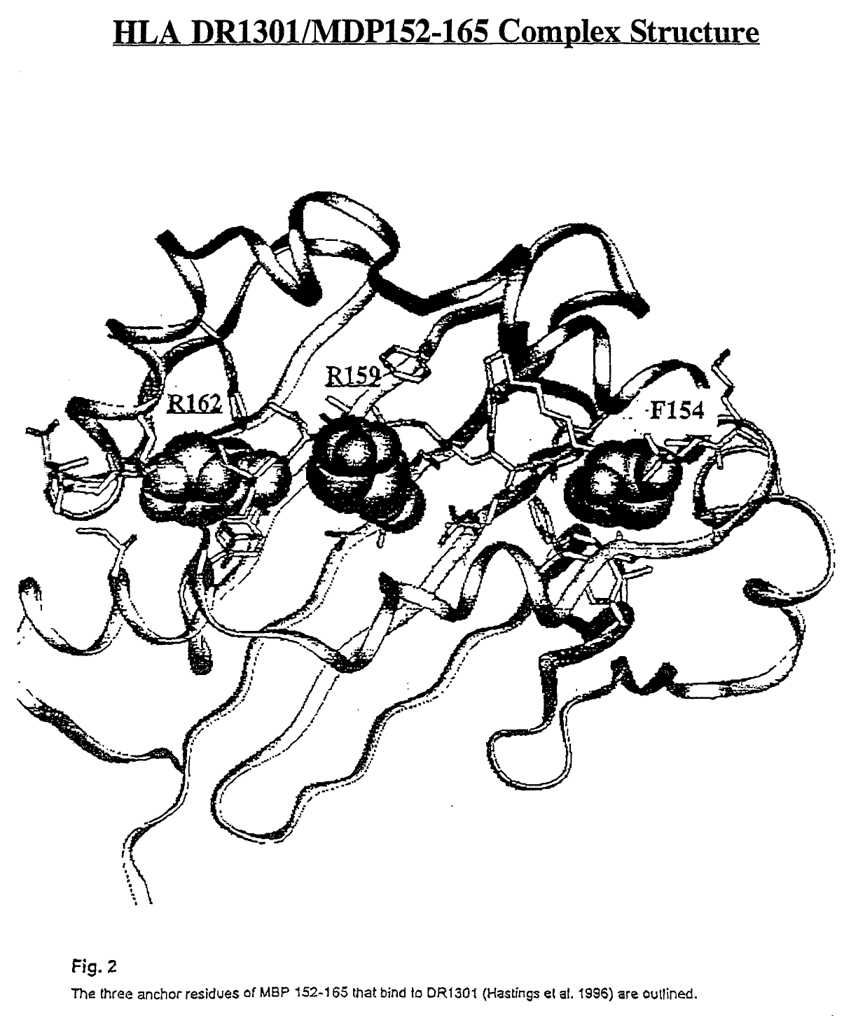

The template structure is further refined by forming a complex including DR-1301 and MBP residues 152-165. As shown in FIG. 2, MBP binds to DR1301 through MBP residues 154 and 162. The pockets communicate through a channel occupied by MBP residue 159. The DR-1301 MBP complex is energy minimized to determine the positions of the side chains forming the binding pockets of DR-1301 in the presence of the MBP anchoring residues.

After a minimum energy structure of the MBP-DR1301 complex is obtained, the MBP residues are removed from the complex and the structure of DR-1301 is maintained rigid for the further stages of the computational processing protocol. That is, computational based prediction of the binding affinity of non proteinaceous compounds to DR-1301 is conducted while maintaining the atoms of DR-1301 in fixed positions obtained by refining the DR-1301 MBP complex.

The structure of DR-1301 is incorporated in the program DOCK, and the NCI database is searched for compounds having adequate geometrical fit with the receptor site of DR-1301. Of the 150,000 compounds in the NCI database, 10,000 compounds are identified as having acceptable geometric fit with the receptor site of DR-1301. The structures of the 10,000 compounds were retrieved from the database and stored in a geometry fit group.

For each compound in the geometry fit group, a complex with DR-1301 is formed by disposing the compound in the receptor site of DR-1301. The structure of the complex is refined through the program MCDOCK. As described above, the program adjusts the positions of the compound inside the receptor site according to an algorithm for the minimization of an energy function describing the interactions between the compound and DR-1301. The energy function includes Van der Waals and electrostatic terms. As discussed above, the only constraints imposed during the minimization relate to the bond lengths and bond angles between bonded atoms, which remain essentially constant during the minimization. In particular, the minimization techniques implemented in MCDOCK allow the atoms to move around rotatable bonds, which allows the compounds to adopt a great number of conformations within the receptor site. The inclusion of the flexibility terms is particularly advantageous in docking flexible molecules into a receptor site.

As discussed in relation to FIG. 2, the receptor site of DR-1301 has a complicated structure, formed by two pockets communicating through a channel. Introducing torsional flexibility in minimizing the structure of the complex allows the compound to probe a larger number of positions in the receptor site. This in turn enhances the probability for identifying a complex structure corresponding to a global minimum of the energy function.

The processing of the compounds in the geometry fit group with the program MCDOCK allows for ranking the compounds based on the minimum energy obtained for the compound-DR1301 complex. An energy based group is formed by including compounds corresponding to complexes having a minimum energy rank of 150 or higher.

The complexes obtained through MCDOCK processing are visualized on a computer screen through the molecular graphics package QUANTA. Compounds having a low complex energy but a poor visual fit to the receptor site are dropped from further consideration.

Based on visual inspection, a group of 106 compounds is formed for in vitro testing. The 106 compounds satisfy both the energy requirement set forth in the MCDOCK protocol and the visual fit requirement described above. It should be noted that 70% of the compounds indicated by MCDOCK as having a high predicted affinity to DR-1301 present adequate visual fit inside the receptor site.

In order to show the advantages provided by the energy processing through the program MCDOCK, the group of 10,000 compounds obtained through the geometry fit processing are processed by the program DOCK. The procedure is similar to the above energy processing of the compound-DR1301 complexes. However, the energy function used in DOCK does not include torsional flexibility terms. Specifically, the DOCK energy function is limited to non-bonded interactions between the atoms of the compound and those of DR-1301. In this procedure, the compound is moved inside the receptor site in a rigid conformation.

A rigid body energy minimization group is formed by compounds corresponding to complexes having a minimized energy rank of 200 or higher. The 200 complexes corresponding to the rigid body minimized structure are individually visualized on a computer screen and the above visual inspection of the complexes is conducted. The visual inspection provides a group of 108 compounds, that is, only 54% of the compounds indicated by the program DOCK as having a affinity to DR-1301 present adequate visual fit inside the receptor site.

While the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the following claims, including equivalents thereof.

### EXAMPLE 2

In Vitro Testing of the 106 Compounds

Material and Methods

The following materials and methods were used for assessing in vitro toxicity, and HLA binding by compounds identified by the described methods.

### Cell Lines

The murine T-cell hybridoma cell line BW58α-β- (a gift of B. Malissen and W. Born) was used for transfection of human TCRα and β cDNA isolated from patients with multiple sclerosis (Hastings, 1996). The parental DAP.3 murine fibroblast cell line was used for transfection of DR(α,β1*1301) or DR((α,β*1501) as previously described (a gift of C. Hurley and R. Rosen-Bronson)) ([Hurley, 1995), [Posch, 1995), [Rosen-Bronson, 1991). All cells lines were maintained in DMEM supplemented with 10% fetal calf serum, 50 U/ml penicillin G, 50 μg/ml streptomycin, 1 mM sodium pyruvate, 2 mM glutamine, and 10 mM HEPES buffer (all Gibco/BRL, Gaithersburg, Md.). TCR- and HLA-transfectants were expanded using 1 mg/ml G418 (Gibco/BRL), or 1 mg/ml G418 and 1 mg/ml hygromycin (Calbiochem, San Diego, Calif.), respectively.

### Cytoflorometric Analysis

Expression of mCD3ε, hVβ22 by BW58α-β- cells and HLA-DR by Dap.3 cells was monitored by flow cytometry (FACStarPlus, Becton-Dickinson, Mountainview, Calif.) using monoclonal antibodies to mCD3ε(PE-labelled Gibco/BRL), hTCRBV22S1 (unlabeled) (Immuntech, Westbrook, Md.), and to HLA-DR (FITC-labelled, Becton-Dickinson, Mountainview, Calif.), respectively. Labeled isotype-matched control Abs (T-Cell Sciences, Cambridge, Mass.) were used to exclude non-specific binding.

### Functional Activation and Blocking Assays

Cells were washed and resuspended in DMEM complete medium without antibodies. 10⁵ cells of the TCR-transfected or untransfected (control) BW58α-β- cells and an equal number of DR-transfected or untransfected (control) Dap.3 cells were incubated with MBP 152-165, or MBP 83-97 and various concentrations of compound, using an U-bottom 96 well plate (Costar). Cell culture supernatants were harvested after two days and stored at −70° C. until use. IL-2 cytokine concentrations (pg/ml) of supernatants were measured by ELISA (Genzyme, Cambridge, Mass.) according to the manufacturer's instructions.

### Peptide-Binding Assays

1.5×10⁶ HLA-DR-transfected Dap.3 cells were washed in Hanks' Balanced Salt Solution, fixed with 1% paraformaldehyde, washed with RPMI medium and PBS, and resuspended in binding buffer. Biotinylated MBP peptide was added at 200 μg/ml (for MBP 152-165) or 1 μg/ml (for MBP 83-97). BSA was added at 1%. The blocking compound (analogue 6) was added at multiple concentrations ranging from 6.25 μM to 400 μM and incubated overnight in a shaking water bath at 37° C. The samples were then washed and lysed with 100 μl lysis buffer containing 1% nonidet-P40 on ice for 40 min. After centrifugation, the supernatant containing the cell membrane fragments was transfected to a 96-well plate pre-coated with the L243 monoclonal antibody that binds to all HLA-DR molecules. The lysis buffer was neutralized with 100 μl n-octyl-β-D-glucopyranoside overnight at 4° C. The samples were then washed and incubated with streptavidin-peroxidase for 1 hour at room temperature. Substrate was added for 10 minutes followed by the stop solution. OD was measured by an ELISA reader at 450 nm. Competition of the compound for the MBP peptide was calculated according to the following formula:

$$\% inhibition = 100 \% = \frac{{OD}_{with compound} - {OD}_{background}}{{OD}_{without compound} - {OD}_{background}} \times 100 \%$$

### Results

When screened by the IL2-production assay described above, eight of the compounds obtained using the program DOCK blocked antigen presentation by DR-1301, but had a similar effect on DR-1501. By contrast, of the eight compounds identified by use of MCDOCK that bound to DR-1301 as evidenced by their activity in functional assays that measured IL-2 secretion by DR1301, restricted TCR transfectants, three demonstrated some degree of specificity for DR-1301 as compared to responses by DR1501-restricted TCR transfectants in the presence of MBP peptide. The results obtained with some of the tested compounds are discussed further in the following examples.

### EXAMPLE 3

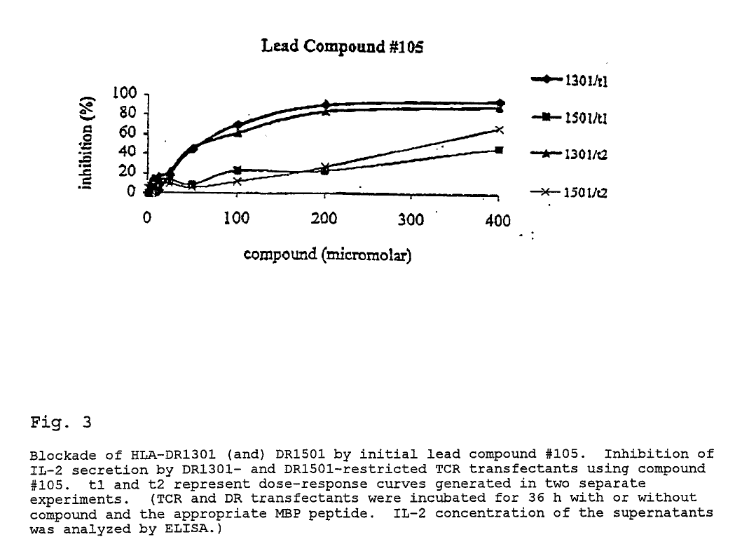

The activity of compounds identified by MCDOCK in functional assays that measure inhibition of IL-2 secretion by DR1301 and DR1501-restricted TCR transfectants using compound #105 and several other compounds was evaluated. The results obtained with compound #105 are contained in FIG. 3. In the figure t1 and t2 represent dose-response curves generated in two different experiments. TCR and DR transfectants were incubated for 36 hours with or without compound and the appropriate MBP peptide. IL-2 concentration of the supernatants was then analyzed by ELISA.

Of the compounds screened, compound #105 was the most effective in discriminating between DR-1301 (strong inhibition) and DR-1501 (weaker inhibition) in IL-2 secretion assays.

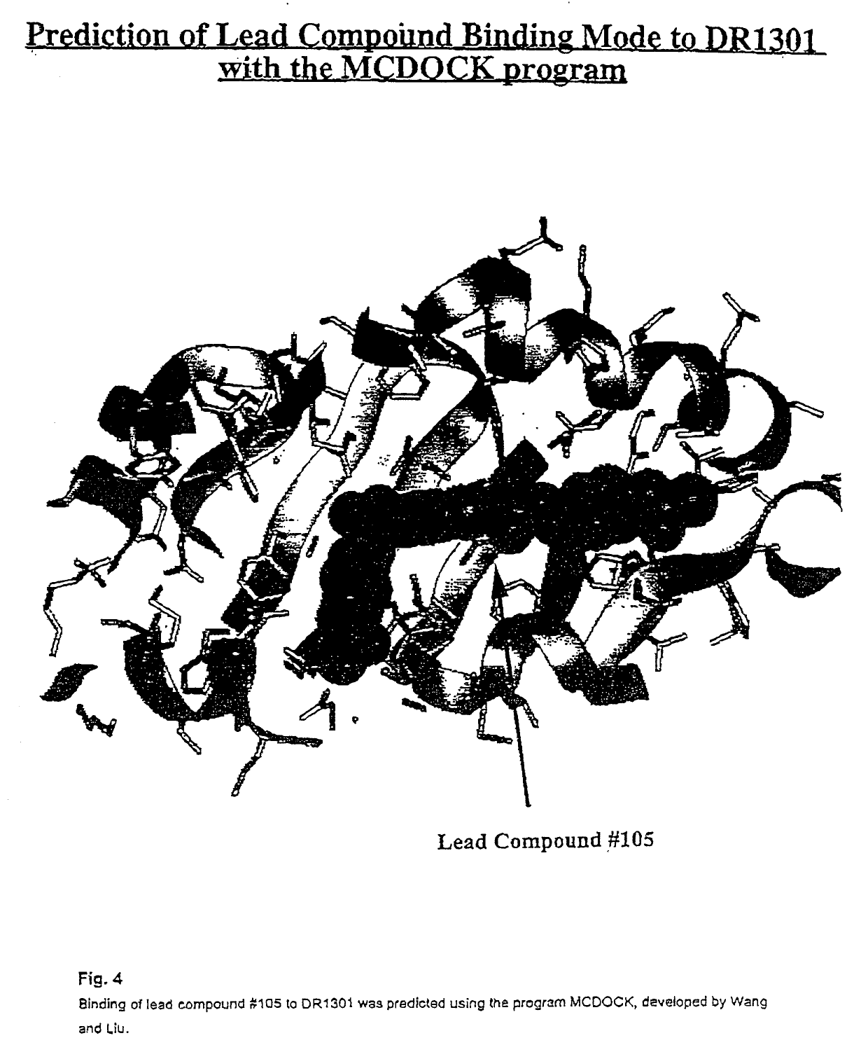

FIG. 4 also contains the predicted binding structure of the tested compound to DR1301 produced according to the MCDOCK program.

### EXAMPLE 4

The specificity of the blocking compounds was tested by reversing their inhibitory effects with increasing concentrations of MBP peptide. In this example, the effect of increasing MBP 152-165 peptide concentrations on IL-2 secretion by DR1301-restricted TCR transfectants was evaluated for four compounds, including compound #105. MBP concentrations were varied from 0 to 1200 micrograms/ml.

Compound #105, exhibited the best dose-response curves when IL-2 production was tested in the presence of increasing MBP peptide concentration. By contrast, reversal of another tested compound induced inhibition of IL-2 production by increasing concentrations of MBP but did not follow a good dose-response curve, suggesting that inhibition of IL-2 secretion was not attributable to specific blocking.

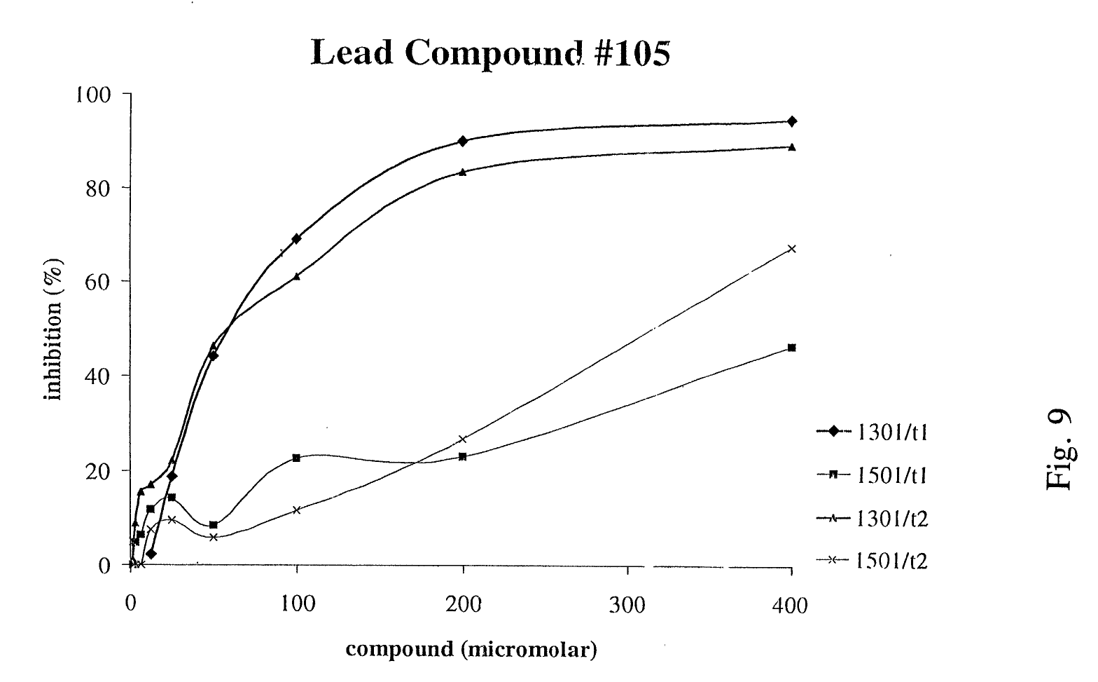

Also, the effect of lead compound #105 on IL-2 secretion in HLA-1301 and 1501 transfectants is contained in FIG. 9. These results further substantiate the significant effect of this particular compound on IL-2 secretion that is likely attributable to its specific binding to HLA-DR1301.

### EXAMPLE 5

Based on the activity of compound #105 in the functional assays, analogs of this compound were selected from the same data base and tested for their effects on IL-2 secretion by DR1301-restricted TCR transfectants.

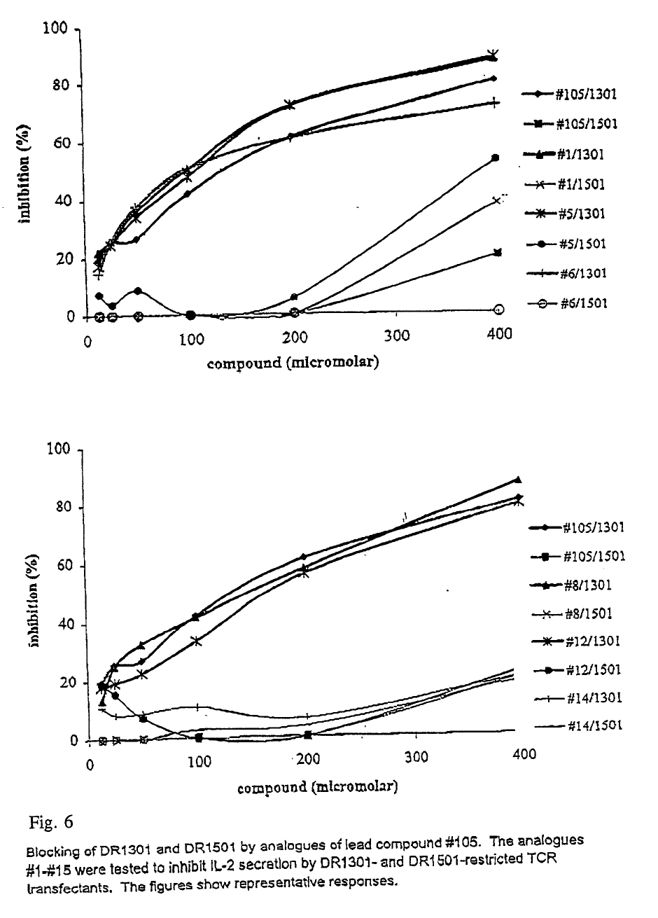

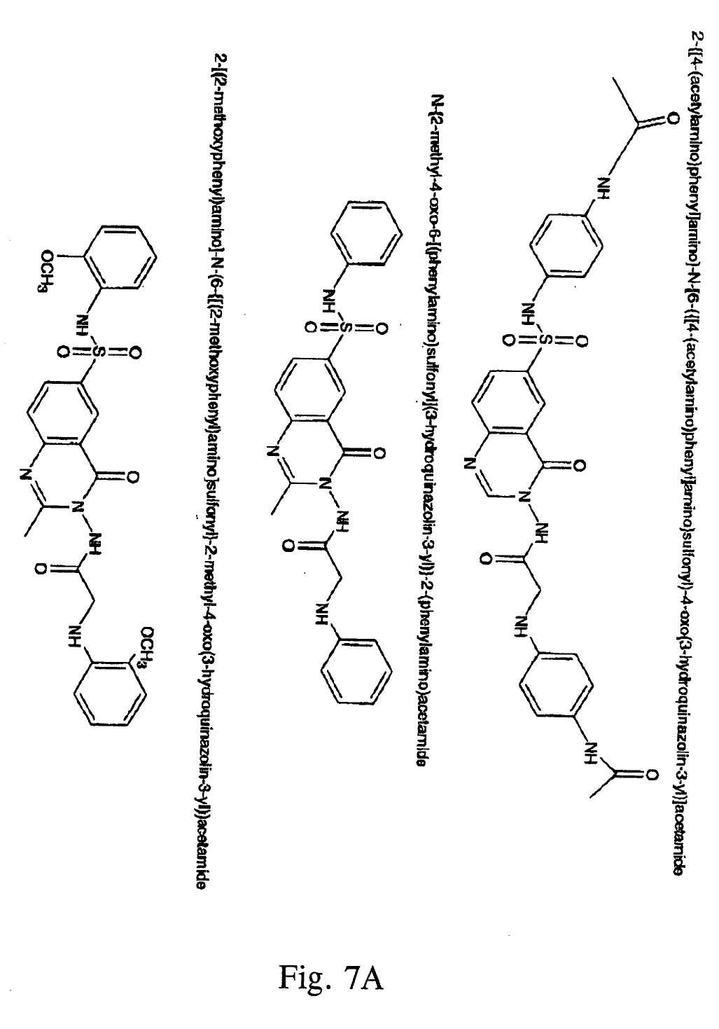

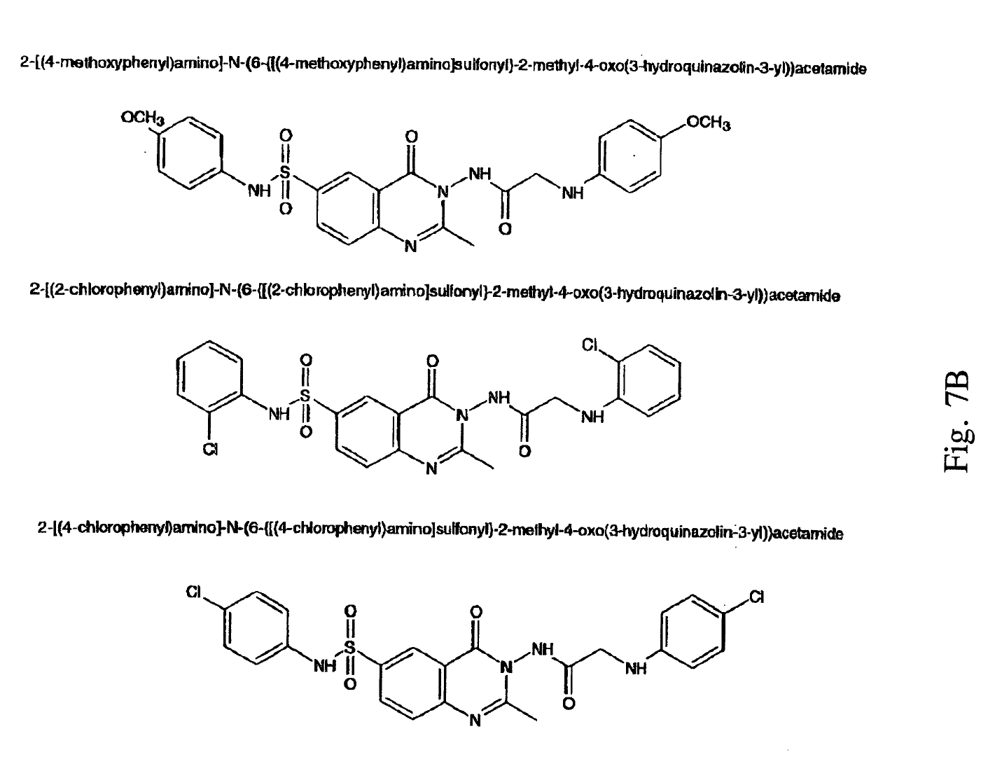

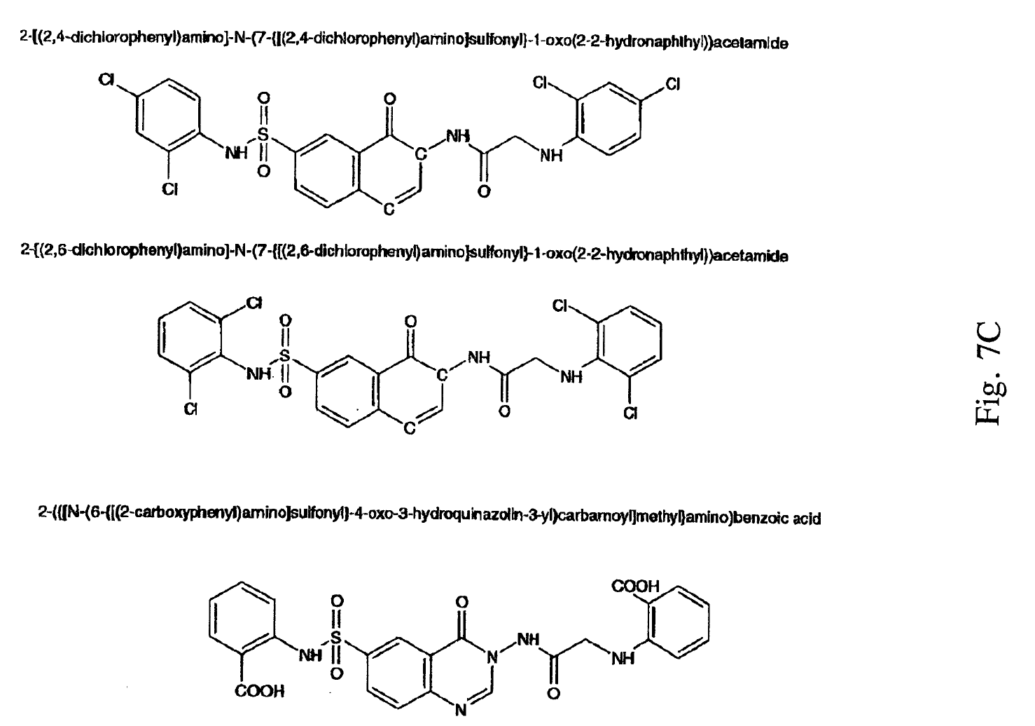

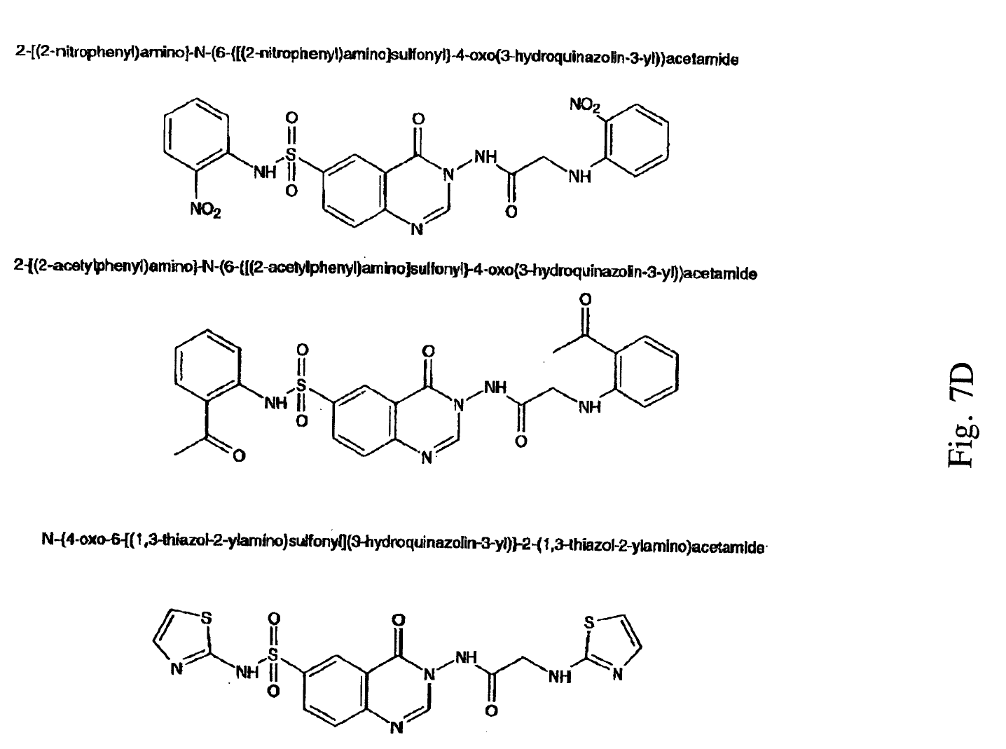

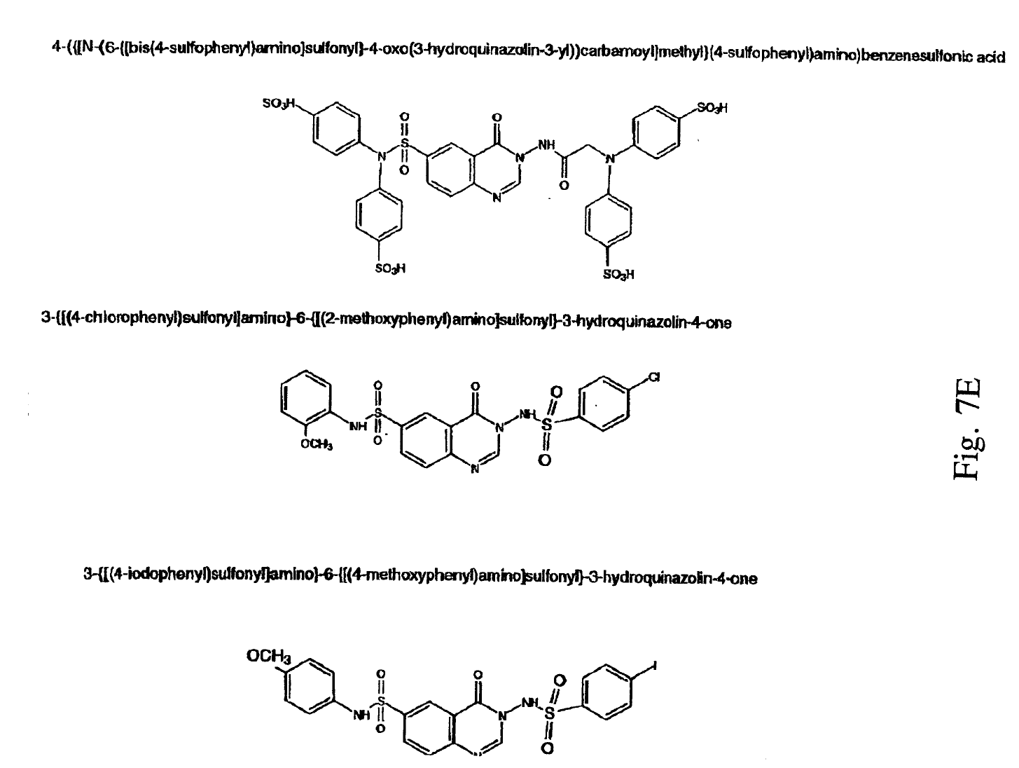

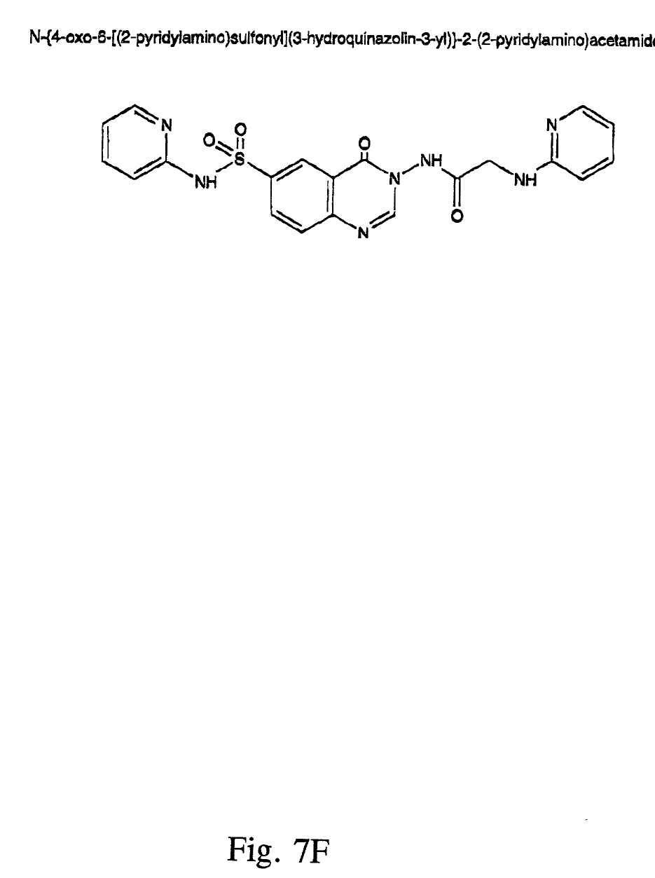

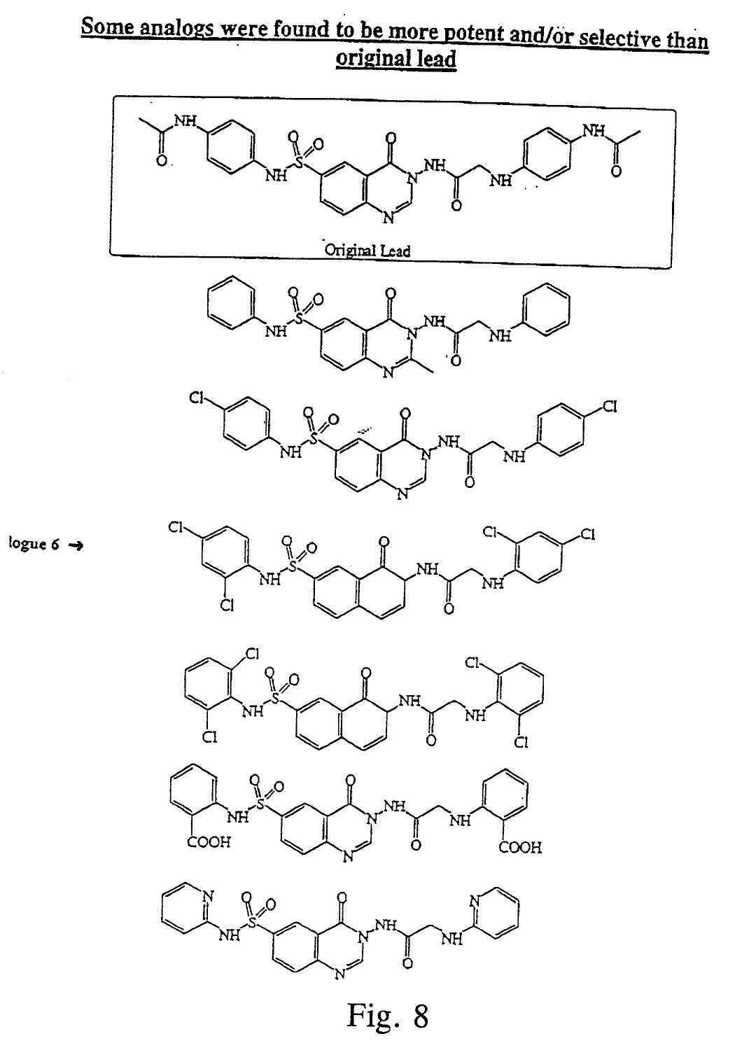

Specifically, the effects of fifteen analogs of compound #105 (referred to as analog #1 to #15) (identified in FIGS. 7A-7F) were compared to the lead compound #105 to assess whether any of such analogs exhibited greater or more specific Il-2 inhibiting activity than the original lead compound. Representative results are contained in FIG. 6. These results indicate that analog #6 exhibited a greater degree of specificity for DR-1301 as compared with DR-1501 when measured by its inhibitory effect on IL-2 secretion, than did the original lead compound.

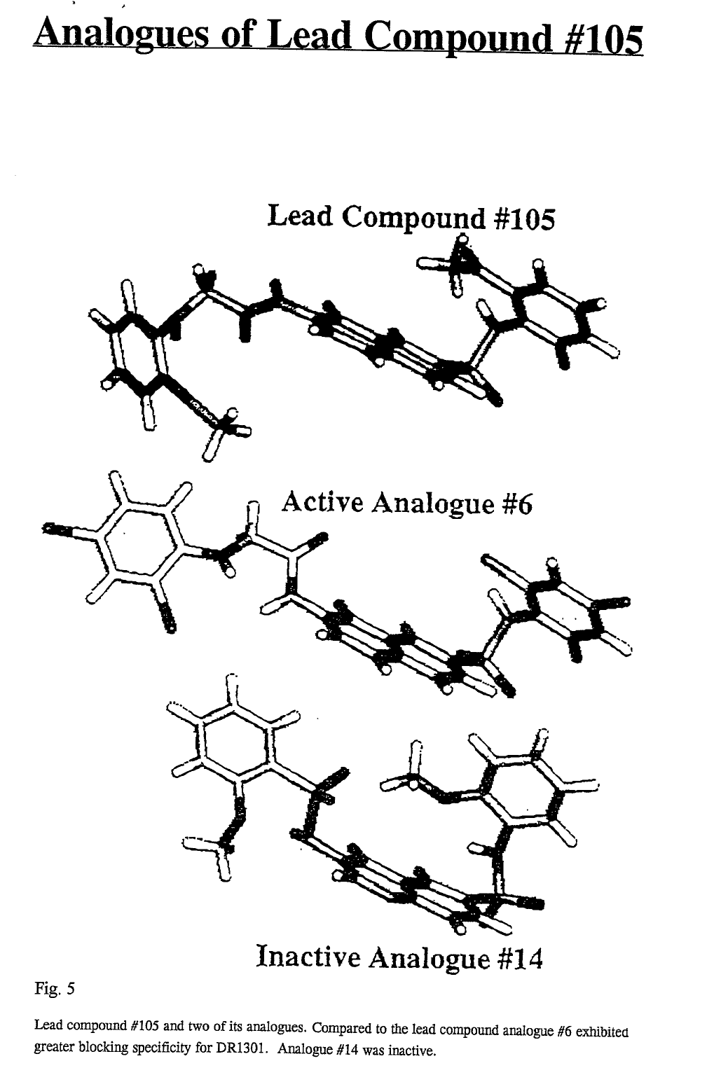

The binding structure for lead compound #105 and analog #6 and inactive analog #14 are contained in FIG. 5. The structures and chemical names for analogs #1 through #15 are contained in FIGS. 7A through 7F.

### EXAMPLE 6

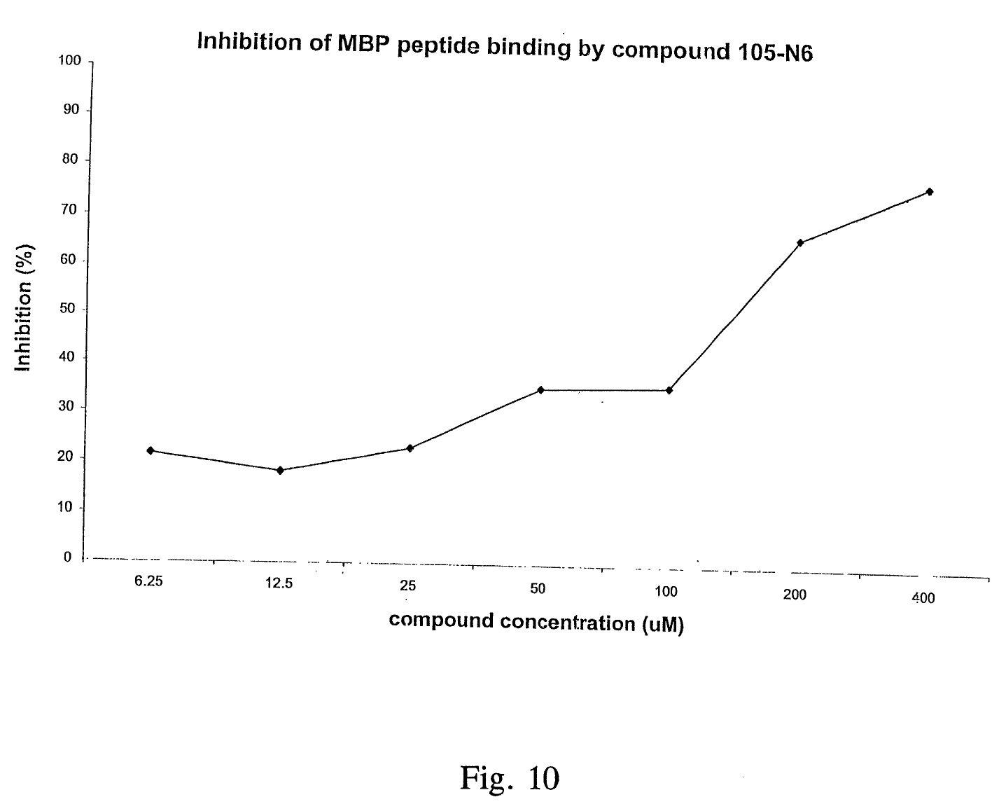

The ability of an analog (#6) 2-[(2,4-dichlorophenyl)amino]-N-(7-{[(2,4-dichlorophenyl)amino]sulfonyl}-1-oxo(2-2-hydronaphthyl))acetamide of lead compound #105 2-{[4-(acetylamino}phenyl]amino)-N-[6-({[4-(acetylamino)phenyl]amino}sulfonyl)-4-oxo(3-hydroquinazolin-3-yl)]acetamide (compound which exhibited the greatest specificity to HLA-DR1301 relative to DR1501 control in the functional assays) was tested in a competitive binding assay. This assay evaluates the binding of biotinylated MBP peptide to HLA-DR1301 in the presence of different concentrations of the analog (analog 6 of lead compound). The amounts of MBP peptide bound (label) to the HLA molecule are then quantitated based on the amount of biotin detected. These results are contained in FIG. 10 and support a conclusion that IL-2 inhibition is most likely attributable to the specific binding of the compound to HLA-DR1301.

While the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the following claims, including equivalents thereof.

What is claimed is:

1. A method for inhibiting the interaction of an HLA molecule to an antigen comprising administering an effective amount of at least one compound having the generic formula: 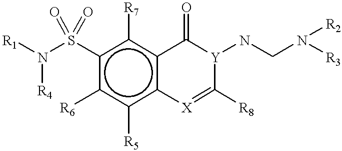
wherein R₁ and R₂ are selected from phenyl, substituted-phenyl, benzyl, substituted-benzyl, or another 5- or 6-membered aromatic ring system, which may optionally contain one or more heteroatoms selected from oxygen, sulfur, and nitrogen, R₃ and R₄ are selected from the group consisting of H, phenyl, substituted-phenyl, benzyl, substituted-benzyl, and other aromatic ring systems, alkyl, preferably C₁ to C₁₀, alkoxy (C₁-C₁₀) halogen, SO₃M (where M is H or alkyl), amide, or COOR where R₁ is H or alkyl;
R₅, R₆, R₇ and R₈ are the same or different and are selected from H, halogen (F, Cl, Br, I), alkyl, alkoxy (C₁-C₁₀), amide, nitro, amine, cycloalkyl (preferably C₁-C₁₀), nitroso, hydroxyl, ether, ester, sulfonic acid, alkenyl, allyl, and X and Y are selected from nitrogen and carbon and may be the same or different.

2. The method of claim 1, wherein said compound is selected from the group consisting of:
Lead Compound #105:
2-{[4-(acetylamino)phenyl]amino}-N-[6-({[4-(acetylamino)phenyl]amino}sulfonyl)-4-oxo(3-hydroquinazolin-3-yl)]acetamide;
Analog 1:
N-{2-methyl-4-oxo-6-[(phenylamino)sulfonyl](3-hydroquinazolin-3-yl)}-2-(phenylamino)acetamide;
Analog 2:
2-[(2-methoxyphenyl)amino]-N-(6-{[(2-methoxyphenyl)amino]sulfonyl}-2-methyl-4-oxo(3-hydroquinazolin-3-yl))acetamide;
Analog 3:
2-[(4-methoxyphenyl)amino]-N-(6-{[(4-methoxyphenyl)amino]sulfonyl}-2-methyl-4-oxo(3-hydroquinazolin-3-yl))acetamide;
Analog 4:
2-[(2-chlorophenyl)amino]-N-(6-{[(2-chlorophenyl)amino]sulfonyl}-2-methyl-4-oxo(3-hydroquinazolin-3-yl))acetamide;
Analog 5:
2-[(4-chlorophenyl)amino]-N-(6-{[(4-chlorophenyl)amino]sulfonyl}-2-methyl-4-oxo(3-hydroquinazolin-3-yl)acetamide;
Analog 6:
2-[(2,4-dichlorophenyl)amino]-N-(7-{[(2,4-dichlorophenyl)amino]sulfonyl}-1-oxo(2-2-hydronaphthyl))acetamide;
Analog 7:
2-[(2,6-dichlorophenyl)amino]-N-(7-{[(2,6-dichlorophenyl)amino}sulfonyl}-1-oxo(2-2-hydronaphthyl))acetamide;
Analog 8:
2-({[N-(6-{[(2-carboxyphenyl)amino]sulfonyl}-4-oxo-3-hydroquinazolin-3-yl)carbamoyl]methyl}amino)benzoic acid;
Analog 9:
2-[(2-nitrophenyl)amino]-N-(6-{[(2-nitrophenyl)amino]sulfonyl}-4-oxo(3-hydroquinazolin-3-yl)acetamide;
Analog 10:
2-[(2-acetylphenyl)amino]-N-(6-{[(2-acetylphenyl)amino]sulfonyl}-4-oxo(3-hydroquinazolin-3-yl))acetamide;
Analog 11:
N-{4-oxo-6-[(1,3-thiazol-2-ylamino)sulfonyl](3-hydroquinazolin-3-yl)}-2-(1,3-thiazol-2-ylamino)acetamide;
Analog 12:
4-({[N-(6-{[bis(4-sulfophenyl)amino]sulfonyl}-4-oxo(3-hydroquinazolin-3-yl))carbamoyl]methyl}(4-sulfophenyl)amino)benzenesulfonic acid;
Analog 13:
3-{[(4-chlorophenyl)sulfonyl]amino}-6-{[(2-methoxyphenyl)amino]sulfonyl}-3-hydroquinazolin-4-one;
Analog 14:
3-{[(4-iodophenyl)sulfonyl]amino}-6-{[(4-methoxyphenyl)amino]sulfonyl}-3-hydroquinazolin-4-one;
Analog 15:
N-{4-oxo-6-[(2-pyridylamino)sulfonyl](3-hydroquinazolin-3-yl)}-2-(2-pyridylamino)acetamide.

3. The method of claim 1, wherein said inhibition results in reduced cytokine production.

4. The method of claim 3, wherein said method results in reduced IL-2 production.

5. A method for treating or preventing a condition involving the interaction of HLA-DR13 (HLA-DR1301) or HLA-DR2 (HLA-DR1501) with an antigen comprising administering an effective amount of at least one compound having generic formula: 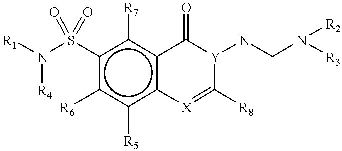
wherein R₁ and R₂ are selected from phenyl, substituted-phenyl, benzyl, substituted-benzyl, or another 5- or 6-membered aromatic ring system, which may optionally contain one or more heteroatoms selected from oxygen, sulfur, and nitrogen, R₃ and R₄ are selected from the group consisting of H, phenyl, substituted-phenyl, benzyl, substituted-benzyl, and other aromatic ring systems, alkyl, preferably C₁ to C₁₀, alkoxy (C₁-C₁₀) halogen, SO₃M (where M is H or alkyl), amide, or COOR where R₁ is H or alkyl;
R₅, R₆, R₇ and R₈ are the same or different and are selected from H, halogen (F, Cl, Br, I), alkyl, alkoxy (C₁-C₁₀), amide, nitro, amine, cycloalkyl (preferably C₁-C₁₀), nitroso, hydroxyl, ether, ester, sulfonic acid, alkenyl, allyl, and X and Y are selected from nitrogen and carbon and may be the same or different.

6. The method of claim 5, wherein said compound is selected from the group consisting of:
Lead Compound #105:
2-{[4-(acetylamino)phenyl]amino}-N-[6-({[4-(acetylamino)phenyl]amino}sulfonyl)-4-oxo(3-hydroquinazolin-3-yl)]acetamide;
Analog 1:
N-{2-methyl-4-oxo-6-[(phenylamino)sulfonyl](3-hydroquinazolin-3-yl)}-2-(phenylamino)acetamide;
Analog 2:
2-[(2-methoxyphenyl)amino]-N-(6-{[(2-methoxyphenyl)amino]sulfonyl}-2-methyl-4-oxo(3-hydroquinazolin-3-yl))acetamide;
Analog 3:
2-[(4-methoxyphenyl)amino]-N-(6-{[(4-methoxyphenyl)amino]sulfonyl}-2-methyl-4-oxo(3-hydroquinazolin-3-yl))acetamide;
Analog 4:
2-[(2-chlorophenyl)amino]-N-(6-{[(2-chlorophenyl)amino]sulfonyl}-2-methyl-4-oxo(3-hydroquinazolin-3-yl))acetamide;
Analog 5:
2-[(4-chlorophenyl)amino]-N-(6-{[(4-chlorophenyl)amino]sulfonyl}-2-methyl-4-oxo(3-hydroquinazolin-3-yl)acetamide;
Analog 6:
2-[(2,4-dichlorophenyl)amino]-N-(7-{[(2,4-dichlorophenyl)amino]sulfonyl}-1-oxo(2-2-hydronaphthyl))acetamide;
Analog 7:
2-[(2,6-dichlorophenyl)amino]-N-(7-{[(2,6-dichlorophenyl)amino}sulfonyl}-1-oxo(2-2-hydronaphthyl))acetamide;
Analog 8:
2-({[N-(6-{[(2-carboxyphenyl)amino]sulfonyl}-4-oxo-3-hydroquinazolin-3-yl)carbamoyl]methyl}amino)benzoic acid;
Analog 9:
2-[(2-nitrophenyl)amino]-N-(6-{[(2-nitrophenyl)amino]sulfonyl}-4-oxo(3-hydroquinazolin-3-yl)acetamide;
Analog 10:
2-[(2-acetylphenyl)amino]-N-(6-{[(2-acetylphenyl)amino]sulfonyl}-4-oxo(3-hydroquinazolin-3-yl))acetamide;
Analog 11:
N-{4-oxo-6-[(1,3-thiazol-2-ylamino)sulfonyl](3-hydroquinazolin-3-yl)}-2-(1,3-thiazol-2-ylamino)acetamide;
Analog 12:
4-({[N-(6-{[bis(4-sulfophenyl)amino]sulfonyl}-4-oxo(3-hydroquinazolin-3-yl))carbamoyl]methyl}(4-sulfophenyl)amino)benzenesulfonic acid;
Analog 13:
3-{[(4-chlorophenyl)sulfonyl]amino}-6-{[(2-methoxyphenyl)amino]sulfonyl}-3-hydroquinazolin-4-one;
Analog 14:
3-{[(4-iodophenyl)sulfonyl]amino}-6-{[(4-methoxyphenyl)amino]sulfonyl}-3-hydroquinazolin-4-one;
Analog 15:
N-{4-oxo-6-[(2-pyridylamino)sulfonyl](3-hydroquinazolin-3-yl)}-2-(2-pyridylamino)acetamide.

7. A method for treating or preventing multiple sclerosis, comprising administrating a therapeutically or prophylactically effective amount of a compound having generic formula: 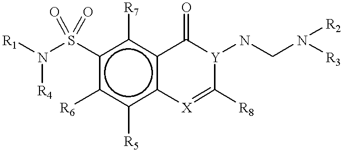
wherein R₁ and R₂ are selected from phenyl, substituted-phenyl, benzyl, substituted-benzyl, or another 5- or 6-membered aromatic ring system, which alkyl, preferably C₁ to C₁₀, alkoxy (C₁-C₁₀) halogen, SO₃M (where M is H or alkyl),amide, or COOR where R₁ is H or alkyl;
R₅, R₆, R₇ and R₈ are the same or different and are selected from H, halogen (F, Cl, Br, I), alkyl, alkoxy (C₁-C₁₀), amide, nitro, amine, cycloalkyl (preferably C₁-C₁₀), nitroso, hydroxyl, ether, ester, sulfonic acid, alkenyl, allyl, and X and Y are selected from nitrogen and carbon and may be the same or different.

8. The method of claim 7, wherein the compound is selected from the group consisting of:
Lead Compound #105:
2-{[4-(acetylamino)phenyl]amino}-N-[6-({[4-(acetylamino)phenyl]amino}sulfonyl)-4-oxo(3-hydroquinazolin-3-yl)]acetamide;
Analog 1:
N-{2-methyl-4-oxo-6-[(phenylamino)sulfonyl](3-hydroquinazolin-3-yl)}-2-(phenylamino)acetamide;
Analog 2:
2-[(2-methoxyphenyl)amino]-N-(6-{[(2-methoxyphenyl)amino]sulfonyl}-2-methyl-4-oxo(3-hydroquinazolin-3-yl))acetamide;
Analog 3:
2-[(4-methoxyphenyl)amino]-N-(6-{[(4-methoxyphenyl)amino]sulfonyl}-2-methyl-4-oxo(3-hydroquinazolin-3-yl))acetamide;
Analog 4:
2-[(2-chlorophenyl)amino]-N-(6-{[(2-chlorophenyl)amino]sulfonyl}-2-methyl-4-oxo(3-hydroquinazolin-3-yl))acetamide;
Analog 5:
2-[(4-chlorophenyl)amino]-N-(6-{[(4-chlorophenyl)amino]sulfonyl}-2-methyl-4-oxo(3-hydroquinazolin-3-yl)acetamide;
Analog 6:
2-[(2,4-dichlorophenyl)amino]-N-(7-{[(2,4-dichlorophenyl)amino]sulfonyl}-1-oxo(2-2-hydronaphthyl))acetamide;
Analog 7:
2-[(2,6-dichlorophenyl)amino]-N-(7-{[(2,6-dichlorophenyl)amino}sulfonyl}-1-oxo(2-2-hydronaphthyl))acetamide;
Analog 8:
2-({[N-(6-{[(2-carboxyphenyl)amino]sulfonyl}-4-oxo-3-hydroquinazolin-3-yl)carbamoyl]methyl}amino)benzoic acid;
Analog 9:
2-[(2-nitrophenyl)amino]-N-(6-{[(2-nitrophenyl)amino]sulfonyl}-4-oxo(3-hydroquinazolin-3-yl)acetamide;
Analog 10:
2-[(2-acetylphenyl)amino]-N-(6-{[(2-acetylphenyl)amino]sulfonyl}-4-oxo(3-hydroquinazolin-3-yl))acetamide;
Analog 11:
N-{4-oxo-6-[(1,3-thiazol-2-ylamino)sulfonyl](3-hydroquinazolin-3-yl)}-2-(1,3-thiazol-2-ylamino)acetamide;
Analog 12:
4-({[N-(6-{[bis(4-sulfophenyl)amino]sulfonyl}-4-oxo(3-hydroquinazolin-3-yl))carbamoyl]methyl}(4-sulfophenyl)amino)benzenesulfonic acid;
Analog 13:
3-{[(4-chlorophenyl)sulfonyl]amino }-6-{[(2-methoxyphenyl)amino]sulfonyl}-3-hydroquinazolin-4-one;
Analog 14:
3-{[(4-iodophenyl)sulfonyl]amino}-6-{[(4-methoxyphenyl)amino]sulfonyl}-3-hydroquinazolin-4-one;
Analog 15:
N-{4-oxo-6-[(2-pyridylamino)sulfonyl](3-hydroquinazolin-3-yl)}-2-(2-pyridylamino)acetamide.

9. The method of claim 8, wherein the compound is 2-{[4-(acetylamino}phenyl]amino)-N-[6-({[4-(acetylamino)phenyl]amino}sulfonyl)-4-oxo(3-hydroquinazolin-3-yl)]acetamide, or 2-[(2,4-dichlorophenyl)amino]-N-(7-{[(2,4-dichlorophenyl)amino]sulfonyl}-1-oxo(2-2-hydronaphthyl))acetamide.

10. The method of claim 8 wherein said therapy includes the administration of another active agent selected from the group consisting of CD40-ligand antagonist, soluble CD40, anti-cytokine antibody, anti-cytokine receptor antibody.

11. A method for processing a compound data base containing three-dimensional structures of chemical compounds to provide a lead compound capable of blocking a receptor site in a host molecule comprising:
modeling a three dimensional structure of the receptor site;
positioning a compound from the compound data base in the receptor site and assigning a geometrical-fit score to said compound indicating the geometrical fit between the structure of said compound and the structure of the receptor site;
ranking the compounds in the data base according to the geometrical-fit score and forming a group of compounds having a geometrical-fit rank of a predetermined value or higher;
minimizing an energy function describing interactions between a compound in the group and the receptor site by adjusting coordinates of said compound to obtain a minimum energy compound-host molecule complex structure;
ranking the compounds in the group according to said minimum energy and forming a first sub-group of compounds having a minimum-energy rank of a predetermined value or higher
visualizing on a computer screen a minimum energy compound-host molecule complex structure and forming a second sub-group of compounds having a visual-fit satisfying a predetermined criterion.

12. The method of claim 11, wherein said energy function comprises a Van der Waals interaction term and an electrostatic interaction term.

13. The method of claim 12, wherein minimizing said energy function comprises probing said compound's conformational flexibility.

14. The method of claim 13, wherein said minimum energy is a global minimum of said function.

15. The method of claim 11, wherein modeling a three dimensional structure of the receptor site comprises providing a set of three coordinates for each atom of the host molecule defining a position of the center of said atom in a three dimensional referential.

16. The method of claim 15, wherein the host molecule is a protein having a known primary structure defined by a sequence of amino-acids forming the protein, known secondary structure, and unknown tertiary structure; and wherein providing a position of the center of each atom of the host molecule comprises:
aligning said sequence of the host molecule with a sequence of a homologous protein obtained from a database of proteins having a known tertiary structure,
assigning a sequence-homology score to each homologous protein indicating the percentage of amino-acids occupying identical positions in the sequence of the host molecule and the sequence of the homologous protein, and forming a template tertiary structure of the host molecule by overlaying atoms of a backbone of corresponding atoms of a host molecule on the backbone atoms of a homologous protein having a sequence-homology score of a predetermined value or higher, and overlaying atoms of a side chain of the host molecule having an equivalent side chain in said homologous protein on corresponding atoms in said homologous protein;
refining said template tertiary structure by adjusting the positions of atoms in a side chain of the host molecule not having an equivalent side chain in said homologous protein to provide a refined tertiary structure having a low energy value defined by an internal energy function describing interactions between the atoms of the host molecule in said refined tertiary structure.

17. The method of claim 16, wherein refining the template tertiary structure comprises positioning a template compound in the receptor site, said template compound having known binding properties to the host molecule and adding to said internal energy function a term describing interactions between said template compound and a side chain of the host molecule.

18. The method of claim 17, wherein said energy function comprises a Van der Waals interaction term and a coulombic interaction term.

19. The method of claim 18, wherein minimizing said energy function comprises probing said compound's conformational flexibility.

20. The method of claim 19, wherein said minimum energy is a global minimum of said function.

21. The method of claim 19, wherein the host molecule is HLA-DR-1301, and said homologous protein is DR1 having a tertiary structure defined by an X-ray structure of DR1 complexed with an influenza peptide.

22. The method of claim 21, wherein said receptor site comprises a negatively charged pocket and a hydrophobic pocket, said template compound comprises the side chains of residues 154 and 162 of Myelin Basic Protein, and wherein the side chain of said residue 154 is disposed in said hydrophobic pocket and said residue 162 is disposed in said negatively charged pocket.

23. The method of claim 22 which further includes testing the identified compounds for their ability to specifically bind to HLA-DR1301 and selecting those compounds having the greatest affinity to HLA-DR1301.

24. The method of claim 23 which further comprises obtaining analogs of said selected compounds, and comparing these compounds in an in vitro assay that measures HLA-DR1301 binding, and selecting for in vivo usage those compounds having the greatest affinity to HLA-DR1301.

25. The method of claim 11, wherein disposing said compound in said receptor site comprises docking said compound.
